(19) United States
(12) Patent Application Publication
(10) Pub. No.: US 2012/0046315 A1
(43) Pub. Date: Feb. 23, 2012

(54) INTERMEDIATE AND ORAL ADMINISTRATIVE FORMATS CONTAINING LENALIDOMIDE

(76) Inventors: Katrin Rimkus, North Rhine-Westphalia (DE); Frank Muskulus, Baden-Wurttemberg (DE); Sandra Brueck, Bavaria (DE); Jana Paetz, North Rhine-Westphalia (DE)

(21) Appl. No.: 13/128,943

(22) PCT Filed: Nov. 13, 2009
(86) PCT No.: PCT/EP09/08105

§ 371 Date: Sep. 26, 2011

(30) Foreign Application Priority Data
Nov. 14, 2008 (DE) 10 2008 057 284.5
Nov. 14, 2008 (DE) 10 2008 057 285.3
Nov. 14, 2008 (DE) 10 2008 057 335.3

(51) Int. Cl. A61K 31/454; A61P 35/00; C07D 401/04

(52) U.S. Cl. 514/323; 546/277.1

(57) ABSTRACT

The invention relates to non-crystalline lenalidomide in the form of a storage-stable intermediate, i.e. preferably amorphous lenalidomide together with a surface stabiliser in the form of a stable intermediate or a storage-stable intermediate, containing lenalidomide and matrix material, wherein the lenalidomide is present in the form of a solid solution (i.e. molecularly disperse). The invention further relates to methods of producing stable amorphous or molecularly disperse lenalidomide and pharmaceutical formulations containing stable amorphous or molecularly disperse lenalidomide. In a second aspect, the invention advantageously relates to dry-processing methods for lenalidomide, especially amorphous and disperse lenalidomide.

The invention relates to non-crystalline lenalidomide in the form of a storage-stable intermediate, i.e. preferably amorphous lenalidomide together with a surface stabiliser in the form of a stable intermediate or an intermediate, containing lenalidomide and matrix material, wherein the lenalidomide is present in the form of a solid solution (i.e. molecularly disperse). The invention further relates to methods of producing stable amorphous or molecularly disperse lenalidomide and pharmaceutical formulations containing stable amorphous or molecularly disperse lenalidomide. In a second aspect, the invention relates to advantageous dry processing methods for lenalidomide, especially amorphous and molecularly disperse lenalidomide.

Lenalidomide is an immune modulator with a variety of effects. It inhibits the proliferation of certain haematopoietic tumour cells, promotes the immunity mediated by T-cells and natural-killer (NK) cells, stimulates erythropoiesis, inhibits angiogenesis and the production of pro-inflammatory cytokines such as TNF-α and interleukin-6 and 12. Lenalidomide is approved for administration to patients with multiple myeloma. Multiple myeloma is a malignant tumour of the B-lymphocytes. Despite chemotherapy and radiation therapy, stem cell transplants and the use of thalidomide and bortezomib, the disease has so far been regarded as incurable in the field.

The IUPAC name for lenalidomide [INN] is 3-(4-amino-1,3-dihydro-1-oxo-2H-isoindol-2-yl)-2,6-piperidine dione. The chemical structure of lenalidomide is shown in formula (1) below:

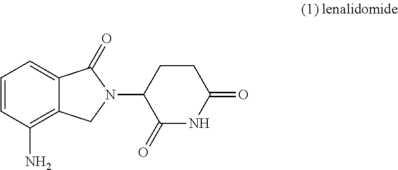

The term “lenalidomide” here comprises both the (R) and the (S) enantiomers.

Synthesis pathways for lenalidomide have been described by Müller et al, Bioorganic & Medicinal Chemistry Letters 9 (1999), 1625-1630, in EP 0 925 294 B1 and in WO 2006/028964. The preparation results in a crystalline solid, and according to WO 2005/023192 eight different polymorphous forms (forms A to H) exist

Lenalidomide is marketed under the trade name Revlimid® as a hard gelatine capsule. Revlimid® contains lenalidomide in crystalline form and is marketed in the form of hard gelatine capsules with 5, 10, 15 and 25 mg lenalidomide. The 5 mg capsule has a content of active agent of approx. 2.5% by weight. In order to ensure the necessary uniformity of the content (=content uniformity), crystalline lenalidomide has to be used in micronised form (see EMEA “Scientific Discussion” for Revlimid, 2007).

The micronisation of lenalidomide entails a number of disadvantages, however. First of all, micronisation results in an active agent with undesirably poor flowability. In addition, the high toxicity makes the micronised active agent more difficult and complicated to handle from the point of view of health and safety. The considerable enlargement of the surface area during micronisation also causes the sensitivity of the active agent to oxidation to increase.

The objective of the present invention was therefore to overcome the above-mentioned disadvantages. The intention is to provide the active agent in a form possessing good flowability and thus making it possible for it to be processed not only into capsules, but also to ensure good compression into tablets. It is also the intention to provide the active agent in a form which does not have a tendency to agglomerate. In addition, it is intended to enable an even distribution of the active agent. It is intended to avoid micronisation of the active agent.

In addition, the intention is to provide lenalidomide in a form that makes it possible to achieve a high level of uniformity of the content (content uniformity), especially with a low content of active agent (drug load).

While developing lenalidomide formulations, the inventors of the present application were also confronted with the fact that crystalline lenalidomide can exist in different polymorphous forms. As described in WO 2005/023192, these polymorphs are frequently not stable, however, but tend to change into different polymorphous forms. For example, the lenalidomide hemihydrate (=form B), which is frequently used, can change into form A or form E under the influence of heat or in a moist environment. As described in WO 2005/023192, however, forms A, B and E have different solubility profiles.

In a patient, the different solubility profile leads to an undesirable, uneven rise in the concentration of the active agent. It was therefore an object of the present invention to provide lenalidomide in a form enabling as even a rise as possible in the concentration in the patient. The aim was largely to avoid both inter-individual and also intra-individual deviations.

The intention is also to provide the active agent in a form which possesses good solubility with good storage stability at the same time.

The evenness of the solubility profile for lenalidomide-formulations is important in this context, especially because of the narrow therapeutic breadth of lenalidomide.

It was unexpectedly possible to solve the problems by converting lenalidomide, especially crystalline lenalidomide, into a stabilised, non-crystalline state. In particular, it was possible to solve the problems by converting lenalidomide into a stabilised amorphous or molecularly disperse state.

The subject matter of the invention is therefore an intermediate containing micronised lenalidomide and a surface stabiliser. The intermediate is amorphous lenalidomide in stabilised form.

The subject matter of the invention is also an intermediate containing lenalidomide and matrix material, wherein the lenalidomide is present in the form of a solid solution. The intermediate is a solid solution of lenalidomide in stabilised form. In the solid solution, lenalidomide is distributed in a “molecularly disperse” manner.

The expressions “surface stabiliser” and “matrix material” are used in the context of this invention to describe stabilised lenalidomide in amorphous form or in the form of a solid solution. The term “surface stabiliser” is preferably used here whenever intermediate of the invention containing amorphous lenalidomide is described. The term “matrix material” is accordingly preferably used whenever intermediate of the invention containing molecularly disperse lenalidomide is described. As will be demonstrated below, the “surface stabiliser” and “matrix material” are preferably identical substances or classes of substances (despite the different designations). In this case, the term “surface stabiliser, or matrix material” is then used.

The subject matter of the invention is also various methods of producing stabilised amorphous lenalidomide, or stabilised molecularly disperse lenalidomide, in the form of the intermediate of the invention.

Finally, the subject matter of the invention comprises pharmaceutical formulations containing the amorphous or molecularly disperse lenalidomide in accordance with the invention or the stabilised lenalidomide of the invention in the form of the intermediate.

In the context of this invention, the term “lenalidomide” comprises 3-(4-amino-1,3-dihydro-1-oxo-2H-isoindol-2-yl)-2,6-piperidine dione in accordance with formula (1) above. In addition, the term “lenalidomide” comprises all the pharmaceutically acceptable salts and solvates thereof. The salts may be acid addition salts. Examples of suitable salts are hydrochlorides, carbonates, hydrogen carbonates, acetates, lactates, butyrates, propionates, sulphates, hydrogen sulphates, methane sulphonates, citrates, tartrates, nitrates, sulphonates, oxalates and/or succinates.

The term “amorphous” is used in the context of this invention to designate the state of solid substances in which the components (atoms, ions or molecules, i.e. in the case of amorphous lenalidomide the lenalidomide molecules) do not exhibit any periodic arrangement over a great range (=long-range order). In amorphous substances, the components are usually not arranged in a totally disordered fashion and completely randomly, but are rather distributed in such a way that a certain regularity and similarity to the crystalline state can be observed with regard to the distance from and orientation towards their closest neighbours (=short-range order). Amorphous substances consequently preferably possess a short-range order, but no long-range order. In addition, an amorphous substance, especially amorphous lenalidomide, usually has an average particle size of more than 300 nm.

In contrast to anisotropic crystals, solid amorphous substances are isotropic. Normally, they do not have a defined melting point, but instead gradually pass over into the liquid state after slowly softening. They can be distinguished from crystalline substances experimentally by means of X-ray diffraction, which does not reveal clearly defined interferences for them, but rather, in most cases, only a few diffuse interferences with small diffraction angles.

The stabilised amorphous lenalidomide used in the context of this invention may consist of amorphous lenalidomide. Alternatively, it may also contain small amounts of crystalline lenalidomide components, provided that no defined melting point of crystalline lenalidomide can be detected in DSC. A mixture containing 90 to 99.99% by weight amorphous lenalidomide and 0.01 to 10% crystalline lenalidomide is preferred, more preferably 95 to 99.9% by weight amorphous lenalidomide and 0.1 to 5% crystalline lenalidomide.

The term “solid solution” is to be understood in the context of this invention as meaning that lenalidomide is distributed in a molecularly disperse manner in a matrix which is present in a solid aggregate state at 25° C.

It is preferable that the intermediate of the invention (containing lenalidomide in the form of a solid solution) contains substantially no crystalline or amorphous lenalidomide. In particular, the intermediate of the invention contains less than 15% by weight, more preferably less than 5% by weight, of amorphous or crystalline lenalidomide, based on the total weight of the lenalidomide present in the intermediate.

It is further preferred that “molecularly disperse” should be understood as meaning that the intermediate of the invention does not contain any lenalidomide particles with a particle size greater than 300 nm, more preferably greater than 200 nm, especially greater than 100 nm. The particle size is determined in this connection by means of confocal Raman spectroscopy. The measuring system preferably consists of an NTEGRA-Spektra Nanofinder ex NT-MDT.

In the context of this invention, the lenalidomide of the invention is present in stabilised form, preferably in stabilised non-crystalline form, two embodiments being preferred:

In a first embodiment, the intermediate is present in a form containing amorphous lenalidomide and a surface stabiliser. In particular, the intermediate of the invention consists substantially of amorphous lenalidomide and surface stabiliser. If—as described below—a crystallisation inhibitor is used in addition, the intermediate of the invention may consist substantially of amorphous lenalidomide, surface stabiliser and crystallisation inhibitor. The expression “substantially” in this case indicates that small amounts of solvent etc. may also be present where applicable.

The surface stabiliser is generally a substance which is suitable for stabilising lenalidomide in amorphous form. The surface stabiliser is preferably a polymer. In addition, the surface stabiliser also includes substances which behave like polymers. Examples of these are fats and waxes. Furthermore, the surface stabiliser also includes solid, non-polymeric compounds which preferably contain polar side groups. Examples of these are sugar alcohols or disaccharides. Finally, the term “surface stabiliser” also encompasses surfactants, especially surfactants which are present in solid form at room temperature.

In a second embodiment, the intermediate is present in a form containing a solid solution of lenalidomide and matrix material. In the context of this invention, the solid solution of lenalidomide of the invention is present in stabilised form, namely in the form of an intermediate, containing molecularly disperse lenalidomide and a matrix material. In particular, the intermediate of the invention consists substantially of molecularly disperse lenalidomide and matrix material. If—as described below—a crystallisation inhibitor is used in addition, the intermediate of the invention may consist substantially of molecularly disperse lenalidomide, matrix material and crystallisation inhibitor. The expression “substantially” in this case indicates that small amounts of solvent etc. may also be present where applicable.

The matrix material is generally a substance which is suitable for stabilising lenalidomide in the form of a solid solution. The matrix material is preferably a polymer. In addition, the matrix material also includes substances which behave like polymers. Examples of these are fats and waxes. Furthermore, the matrix material also includes solid, non-polymeric compounds which preferably contain polar side groups. Examples of these are sugar alcohols or disaccharides. Finally, the term “matrix material” also encompasses surfactants, especially surfactants which are present in solid form at room temperature.

A further subject matter of the invention is a method of identifying a pharmaceutical excipient which is suitable as a surface stabiliser for amorphous lenalidomide or as a matrix material for molecularly disperse lenalidomide and which can hence be used for preparing the intermediate of the invention.

The method relating to amorphous lenalidomide comprises the steps of:
a) Providing a pharmaceutical excipient which is present in a solid aggregate state at 25° C. For this purpose, it is generally possible to choose the pharmaceutical excipients mentioned in the European Pharmacopoeia.
b) Twice in succession, heating up the solid excipient by means of DSC. In this case, two heating curves are recorded by means of DSC. The curves are usually recorded from 20° C. to no more than 20° C. below the decomposition range of the substance to be tested.

For this purpose a Mettler Toledo DSC 1 apparatus can be used. The work is performed at a heating rate of 1-20° C./min., preferably 5-15° C./min., and at a cooling rate of 5-25, preferably 10-20° C./min.
c) Selecting the excipient as “suitable” if a glass transition point of 20 to 120° C., preferably 25° C. to 100° C., can be seen in the second DSC heating curve.

The method relating to molecularly disperse lenalidomide comprises the steps of:
a) preparing lenalidomide, a pharmaceutical excipient which is present in a solid aggregate state at 25° C., and a 1:1 mixture of lenalidomide and excipient;
b) twice heating up the solid excipient by means of DSC and identifying the glass transition temperature of the excipient (Tg_{Excip});
c) twice heating up the active agent lenalidomide by means of DSC and identifying the glass transition temperature of the active agent (Tg_{Lena});
d) twice heating up a 1:1 mixture of lenalidomide and excipient by means of DSC and identifying the glass transition temperature of the mixture (Tg_{Mix}), and
e) selecting the excipient as “suitable” provided that Tg_{Mix} is between Tg_{Excip} and Tg_{Lena}.

In this case, two heating curves are recorded by means of DSC. The curves are usually recorded from 20° C. to no more than 20° C. below the decomposition range of the substance to be tested. The term “1:1-mixture” refers to a mixture of 50% by weight lenalidomide and 50% by weight excipient, which is prepared by mixing.

For both cases, a Mettler Toledo DSC 1 apparatus can be used. The work is performed at a heating rate of 1-20° C./min., preferably 5-15° C./min., and at a cooling rate of 5-25, preferably 10-20° C./min.

Another subject matter of the invention is intermediates containing amorphous or molecularly disperse lenalidomide and a pharmaceutical excipient, selected by means of the methods described above.

The surface stabiliser or matrix material used for the preparation of the intermediate of the invention is preferably a polymer. The polymer that can be used for the preparation of the intermediate preferably has a glass transition temperature (Tg) of more than 20° C., more preferably 30° C. to 150° C., especially 40° C. to 100° C. In the case of the solid solution, furthermore, the polymer used as the matrix material preferably has a glass transition temperature (Tg) of more than 25° C., especially more than 35° C. A polymer with an appropriately selected Tg causes immobilisation, which prevents the recrystallisation of the amorphous lenalidomide or the reversion of the molecular lenalidomide dispersion into colloids or particles.

The term “glass transition temperature” (Tg) is used to describe the temperature at which amorphous or partially crystalline polymers change from the solid state to the liquid state. In the process, a distinct change in physical parameters, e.g. hardness and elasticity, occurs. Below the Tg, a polymer is usually glassy and hard, whereas above the Tg, it changes into a rubber-like to viscous state. The glass transition temperature is determined in the context of this invention by means of dynamic differential scanning calorimetry (DSC). For this purpose a Mettler Toledo DSC 1 apparatus can be used. The work is performed at a heating rate of 1-20° C./min., preferably 5-15° C./min., and at a cooling rate of 5-25, preferably 10-20° C./min.

In addition the polymer to be used for the preparation of the intermediate preferably has a number-average molecular weight, more preferably a weight-average molecular weight, of 1,000 to 500,000 g/mol, more preferably from 2,000 to 90,000 g/mol. When the polymer used in the preparation of the intermediate is dissolved in water in an amount of 2% by weight, the resulting solution preferably has a viscosity of 0.1 to 18 mPa/s, more preferably 0.5 to 15 mPa/s, especially 1.0 to 8 mPa/s, measured at 25° C. and preferably determined in accordance with Ph. Eur. 6.0, Chapter 2.2.10. The weight-average molecular weight is determined in the context of this invention by means of gel permeation chromatography.

Hydrophilic polymers are preferably used for the preparation of the intermediate. This refers to polymers which possess hydrophilic groups. Examples of suitable hydrophilic groups are hydroxy, alkoxy, acrylate, methacrylate, sulphonate, carboxylate and quaternary ammonium groups.

The intermediate of the invention may, for example, comprise the following hydrophilic polymers as the surface stabiliser or matrix material: polysaccharides, such as hydroxypropyl methyl cellulose (HPMC), carboxymethyl cellulose (CMC, especially sodium and calcium salts), ethyl cellulose, methyl cellulose, hydroxyethyl cellulose, ethyl hydroxyethyl cellulose, hydroxypropyl cellulose (HPC); microcrystalline cellulose, polyvinyl pyrrolidone, polyvinyl acetate (PVAC), polyvinyl alcohol (PVA), polymers of acrylic acid and their salts, polyacrylamide, polymethacrylates, vinyl pyrrolidone/vinyl acetate copolymers (such as Kollidon® VA64, BASF), polyalkylene glycols, such as polypropylene glycol or preferably polyethylene glycol, co-block polymers of polyethylene glycol, especially co-block polymers of polyethylene glycol and polypropylene glycol (Pluronic®, BASF), and mixtures of the polymers mentioned.

Substances particularly preferably used as surface stabilisers or matrix materials are polyvinyl pyrrolidone, preferably with a weight-average molecular weight of 10,000 to 60,000 g/mol, especially 12,000 to 40,000 g/mol, a copolymer of vinyl pyrrolidone and vinyl acetate, especially with a weight-average molecular weight of 40,000 to 70,000 g/mol and/or polyethylene glycol, especially with a weight-average molecular weight of 2,000 to 10,000 g/mol, and HPMC, especially with a weight-average molecular weight of 20,000 to 90,000 g/mol and/or preferably a content of methyl groups of 10 to 35% and a content of hydroxy groups of 1 to 35%. In addition microcrystalline cellulose can preferably be used, especially one with a specific surface area of 0.7-1.4 m²/g. The specific surface area is determined by means of the gas adsorption method according to Brunauer, Emmet and Teller.

Co-block polymers of polyethylene glycol and polypropylene glycol can likewise preferably be used as surface stabilisers or matrix materials, i.e. polyoxyethylene/polyoxypropylene block polymers. These preferably have a weight-average molecular weight of 1,000 to 20,000 g/mol, more preferably 1,500 to 12,500 g/mol, especially 5,000 to 10,000 g/mol. These block polymers are preferably obtainable by condensation of propylene oxide with propylene glycol and subsequent condensation of the polymer formed with ethylene oxide. This means that the ethylene oxide content is preferably present as an “endblock”. The block polymers preferably have a weight ratio of propylene oxide to ethylene oxide of 50:50 to 95:5, more preferably 70:30 to 90:10. The block polymers preferably have a viscosity at 25° C. of 200 to 2,000 mPas, more preferably 500 to 1,500 mPas, especially 800 to 1,200 mPas.

Furthermore, the surface stabiliser or matrix material also includes solid, non-polymeric compounds which preferably contain polar side groups. Examples of these are sugar alcohols or disaccharides. Examples of suitable sugar alcohols and/or disaccharides are mannitol, sorbitol, xylitol, isomalt, glucose, fructose, maltose and mixtures thereof. The term “sugar alcohols” in this context also includes monosaccharides. In particular, isomalt and sorbitol are used as the surface stabiliser or matrix material.

Alternatively, it is possible to use waxes, such as cetyl palmitate or carnauba wax as the surface stabiliser or matrix material. It is likewise possible to use fats, such as glycerol fatty acid esters (e.g. glycerol palmitate, glycerol behenate, glycerol laurate, glycerol stearate) or PEG glycerol fatty acid esters.

In a preferred embodiment, the intermediate of the invention contains amorphous lenalidomide and surface stabiliser, the weight ratio of lenalidomide to surface stabiliser being 4:1 to 1:50, more preferably 2:1 to 1:20, even more preferably 1:1 to 1:15, especially 1:2 to 1:10.

In a preferred embodiment, the intermediate contains lenalidomide of the invention and matrix material, the weight ratio of lenalidomide to matrix material being 2:1 to 1:100, more preferably 1:1 to 1:50, even more preferably 1:2 to 1:30, especially 1:5 to 1:20, and alternatively particularly preferably 1:2 to 1:10.

It is preferable that that type and quantity of surface stabiliser or matrix material should be selected such that the resulting intermediate a glass transition temperature (Tg) of more than 20° C., preferably >25° C. (especially for intermediates containing molecularly disperse lenalidomide) or preferably >30° C. (especially for intermediates containing amorphous lenalidomide).

It is preferable that the type and quantity of the polymer should be selected such that the resulting intermediate is storage-stable. “Storage-stable” means that in the intermediate of the invention, after storage for 3 years at 25° C. and 50% relative humidity, the proportion of crystalline lenalidomide—based on the total amount of lenalidomide—is no more than 60% by weight, preferably no more than 30% by weight, more preferably no more than 15% by weight, in particular no more than 5% by weight.

It is advantageous for the surface stabiliser or the matrix material to be used in particulate form, wherein the volume-average particle size (D50) is less than 500 μm, preferably 5 to 250 μm.

In a preferred embodiment, in addition to amorphous lenalidomide and surface stabiliser or to molecularly disperse lenalidomide and matrix material, the intermediates of the invention also contain a crystallisation inhibitor based on an inorganic salt, an organic acid or a polymer with a weight-average molecular weight (Mw) of more than 500,000 g/mol. These polymers which are suitable as crystallisation inhibitors are also referred to in the context of this invention as “high-viscosity polymers”. Their weight-average molecular weight is usually less than 5,000,000 g/mol. A preferred high-viscosity polymer is povidone.

The crystallisation inhibitor is preferably ammonium chloride, citric acid, or Povidone K 90 (in accordance with Ph. Eur. 6.0).

The crystallisation inhibitor can generally be used in an amount of 1 to 30% by weight, preferably 2 to 25% by weight, more preferably 5 to 20% by weight, based on the total weight of the intermediate.

The intermediates of the invention are obtainable by a variety of preparation methods. Depending on the preparation method, the intermediates are obtained in different particle sizes. Normally, the intermediates of the invention are present in particulate form and have an average particle diameter (D50) of 1 to 750 μm, depending on the preparation method in each case.

The expression “average particle diameter” relates in the context of this invention to the D50 value of the volume-average particle diameter determined by means of laser diffractometry. In particular, a Malvern Instruments Mastersizer 2000 was used to determine the diameter (wet measurement with ultrasound for 60 sec., 2,000 rpm, the evaluation being performed using the Fraunhofer model), and preferably using a dispersant in which the substance to be measured does not dissolve at 20° C.).

The average particle diameter, which is also referred to as the D50 value of the integral volume distribution, is defined in the context of this invention as the particle diameter at which 50% by volume of the particles have a smaller diameter than the diameter which corresponds to the D50 value. Similarly, 50% by volume of the particles then have a larger diameter than the D50 value.

Another subject matter of the invention is a method of preparing the intermediate of the invention. In the following, five preferred embodiments of such a method will be explained.

In a first embodiment, the invention relates to a freeze-drying method, i.e. a method of preparing the intermediate of the invention, comprising the steps of
(a1) dissolving the lenalidomide, preferably the crystalline lenalidomide and the surface stabiliser or matrix material, in a solvent or mixture of solvents, and
(b1) freeze-drying the solution from step (a1).

In step (a1), lenalidomide, preferably crystalline lenalidomide and the surface stabiliser described above or the matrix material described above, is dissolved, preferably completely dissolved, in a solvent or mixture of solvents.

Suitable solvents are. for example, water, alcohol (e.g. methanol, ethanol, isopropanol), dimethyl sulphoxide (DMSO), acetone, butanol, ethyl acetate, heptane, pentanol or mixtures thereof. Preferably, a mixture of water and ethanol is used.

Suitable surface stabilisers or matrix materials in this embodiment are in particular modified celluloses, such as HPMC, and sugar alcohols, such as isomalt, mannitol and sorbitol.

If the intermediate to be prepared is additionally intended to contain a crystallisation inhibitor based on an inorganic salt or an organic acid, or a highly viscous polymer, this can likewise be added in step (a1). Reference is made to the above statements with regard to the type and amount of the crystallisation inhibitor.

The solution from step (a1) is cooled to about 10 to 50° C. below freezing point (i.e. it is frozen). Then the solvent is removed by sublimation. This is preferably done when the conductivity of the solution is less than 2%. The sublimation temperature is preferably determined by the point of intersection of the product temperature and Rx −10° C. Sublimation is preferably effected at a pressure of less than 0.1 mbar.

After completion of the sublimation, the lyophilised intermediate is heated to room temperature.

The process conditions in this first embodiment are preferably selected such that the resulting intermediate particles have a volume-average particle diameter (D50) of 5 to 250 μm, more preferably 3 to 150 μm, in particular 5 to 100 μm.

In a second preferred embodiment, the invention relates to a melt “pellet-layering process”, i.e. a method of preparing the intermediate of the invention, comprising the steps of
(a2) dissolving the lenalidomide, preferably the crystalline lenalidomide and the surface stabiliser or matrix material, in a solvent or mixture of solvents, and
(b2) spraying the solution from step (a2) onto a substrate core.

In step (a2), lenalidomide, preferably crystalline lenalidomide and the surface stabiliser described above or the matrix material described above, is dissolved, preferably completely dissolved, in a solvent or mixture of solvents.

Suitable solvents are, for example, water, alcohol (e.g. methanol, ethanol, isopropanol), dimethyl sulphoxide (DMSO), acetone, butanol, ethyl acetate, heptane, pentanol or mixtures thereof. Preferably, a mixture of water and ethanol is used.

Suitable surface stabilisers or matrix materials in this second embodiment are in particular modified celluloses, such as HPMC, sugar alcohols, such as isomalt and sorbitol, and polyethylene glycol, especially polyethylene glycol with a molecular weight of 2,000 to 10,000 g/mol.

If the intermediate to be prepared is additionally intended to contain a crystallisation inhibitor based on an inorganic salt or an organic acid, or a highly viscous polymer, this can likewise be added in step (a2). Reference is made to the above statements with regard to the type and amount of the crystallisation inhibitor.

In step (b2), the solution from step (a2) is sprayed onto a substrate core. Suitable substrate cores are particles consisting of pharmaceutically acceptable excipients, especially “neutral pellets”. The pellets preferably used are those which are obtainable under the trade name Cellets® and which contain a mixture of lactose and microcrystalline cellulose, or sugar spheres, which are a mixture of starch and sugar.

Step (b2) is preferably performed in a fluidised bed dryer, such as a Glatt GPCG 3 (Glatt GmbH, Germany). Work is preferably performed with air inlet temperatures of 60 to 80° C., with product temperatures of 30 to 40° C. and with a spray pressure of 1 to 1.5 bar.

The process conditions in this second embodiment are preferably selected such that the resulting intermediate particles have a volume-average particle diameter (D50) of 50 to 800 μm, more preferably 150 to 650 μm.

In a third embodiment, the invention relates to a spray-drying method of preparing the intermediate of the invention, comprising the steps of
(a3) dissolving the lenalidomide, preferably the crystalline lenalidomide and the surface stabiliser or matrix material, in a solvent or mixture of solvents, and
(b3) spray-drying the solution from step (a3).

In step (a3), lenalidomide, preferably crystalline lenalidomide and the surface stabiliser described above or the matrix material described above, is dissolved, preferably completely dissolved, in a solvent or mixture of solvents.

Suitable solvents are. for example, water, alcohol (e.g. methanol, ethanol, isopropanol), dimethyl sulphoxide (DMSO), acetone, butanol, ethyl acetate, heptane, pentanol or mixtures thereof. Preferably, an ethanol/water mixture is used.

Suitable surface stabilisers or matrix materials in this embodiment are in particular modified celluloses, such as HPMC, polyvinyl pyrrolidone and copolymers thereof, and sugar alcohols, such as isomalt and sorbitol, or mixtures thereof. In the case of polymers, it is preferable to use polymers with the molecular weights specified above.

If the intermediate to be prepared is additionally intended to contain a crystallisation inhibitor based on an inorganic salt or an organic acid, or a highly viscous polymer, this can likewise be added in step (a3). Reference is made to the above statements with regard to the type and amount of the crystallisation inhibitor.

In the subsequent step (b3), the solution from step (a3) is spray-dried. The spray-drying is usually carried out in a spray tower. As an example, a Büchi B-191 is suitable (Büchi Labortechnik GmbH, Germany). Preferably an inlet temperature of 100° C. to 150° C. is chosen. The amount of air is, for example, 500 to 700 litres/hour, and the aspirator preferably runs at 80 to 100%.

The process conditions in this third embodiment are preferably selected such that the resulting intermediate particles have a volume-average particle diameter (D50) of 1 to 250 μm, more preferably 2 to 150 μm, especially 3 to 100 μm.

In a fourth preferred embodiment, the invention relates to a melt process, preferably a melt extrusion process i.e. a method of preparing the intermediate of the invention, comprising the steps of
(a4) mixing lenalidomide, preferably the crystalline lenalidomide and surface stabiliser or matrix material, preferably polymeric surface stabiliser or matrix material, and
(b4) melting, preferably extruding the mixture.

Of the six preparation methods described, the fourth embodiment is particularly preferable.

In step (a4), lenalidomide, preferably crystalline lenalidomide, is mixed with the surface stabiliser or the matrix material (preferably in a mixer). In this embodiment of the method of the invention, a surface stabiliser or matrix material in polymeric form is used.

Suitable polymeric surface stabilisers or matrix materials in this fourth embodiment are especially polyvinyl pyrrolidone and vinyl pyrrolidone/vinyl acetate copolymers, and also polyvinyl alcohols, methacrylates and HPMC, preferably with the molecular weights specified above. Similarly, sugar alcohols are preferably used, more preferably selected from isomalt and sorbitol; in particular, isomalt is used as the surface stabiliser or matrix material.

If the intermediate to be prepared is additionally intended to contain a crystallisation inhibitor based on an inorganic salt or an organic acid, or a highly viscous polymer, this can likewise be added in step (a4). Reference is made to the above statements with regard to the type and amount of the crystallisation inhibitor.

In step (b4), the mixture is melted, preferably extruded. For this purpose, conventional melt extruders can be used. By way of example, a Leistritz Micro 18 is used.

The melt-processing (=step b4) can preferably be carried out as melt granulation or melt extrusion.

In a preferred embodiment, melt granulation is performed. In this case, the melting process is preferably performed by means of an intensive mixer with a heatable jacket unit; a Diosna® P1-6, for example, can advantageously be used. In this context, it is usual for the mixture of lenalidomide and surface stabiliser or matrix material to be pre-mixed and only heated up in a second step (e.g. by switching on the heatable jacket), preferably with stirring. The heating is preferably continued until an increase in the power consumption is observed. After that, the mixture is granulated and cooled.

In a preferred embodiment, melt extrusion is performed. This is a continuous method (independent of batches), where the pre-mixing and granulating are not performed sequentially in time, but rather in one production step. A preferred method of preparing the melt extrudate is melt extrusion by means of a twin-screw extruder (e.g. Leistritz® micro 18). The advantage here is the possibility of setting a temperature gradient, depending on the surface stabiliser or matrix material chosen, which allows the dwell time of the lenalidomide at high temperatures to be reduced considerably. The temperature gradient is usually between 40-250° C. and is preferably selected such that after processing, the lenalidomide is no longer present in crystalline form.

The melting temperature, preferably the extrusion temperature, generally depends on the nature of the surface stabiliser or matrix material. It is usually between 40 and 250° C., preferably between 80 and 160° C., especially in the case of amorphous lenalidomide. Alternatively, in the case of molecularly disperse lenalidomide, it preferably lies between 50 and 250° C., more preferably between 100 and 200° C. The extrusion is preferably carried out at an outlet pressure of 10 bar to 100 bar, more preferably at 20 to 80 bar.

The cooled melt is usually comminuted by a rasp screen (e.g. Comill® U5) and in this way accordingly reduced to a uniform particle size.

The process conditions in this fourth embodiment are preferably selected such that the resulting intermediate particles have a volume-average particle diameter (D50) of 150 to 1,000 μm, more preferably a D50 of 250 to 800 μm.

Instead of granulating the extruded material, “direct injection moulding” may also be performed. In this case, the method of the invention includes the step of
(c4) injection moulding the extruded material into moulds for pharmaceutical dosage forms.

Examples are moulds for tablets.

In a fifth embodiment, the invention relates to a milling process, i.e. a method of preparing the intermediate of the invention, comprising the steps of
(a5) mixing lenalidomide, preferably crystalline lenalidomide, and surface stabiliser, and
(b5) milling the mixture from step (a5), the milling conditions preferably being selected such that there is a transition from crystalline to amorphous lenalidomide.

Crystalline lenalidomide and surface stabiliser are preferably mixed in step (a5). The mixture is milled in step (b5). The mixing may take place before or even during the milling, i.e. steps (a5) and (b5) may be performed simultaneously.

If the intermediate to be prepared is additionally intended to contain a crystallisation inhibitor based on an inorganic salt or an organic acid, this can likewise be added in step (a5) or (b5). Reference is made to the above statements with regard to the type and amount of the crystallisation inhibitor.

The milling conditions are preferably selected such that there is a transition from crystalline to amorphous lenalidomide.

The milling is generally performed in conventional milling apparatuses, preferably in a ball mill, such as a Retsch PM 100.

The milling time is usually 10 minutes to 10 hours, preferably 30 minutes to 8 hours, more preferably 2 hours to 6 hours.

Suitable surface stabilisers in this fifth embodiment are in particular polyvinyl pyrrolidone, modified celluloses, such as HPMC, sugar alcohols, such as isomalt and sorbitol, and polyethylene glycol, especially polyethylene glycol with a molecular weight of 2,000 to 10,000 g/mol.

The process conditions in this fifth embodiment are preferably selected such that the resulting intermediate particles have a volume-average particle diameter (D50) of 1 to 350 μm, more preferably 10 to 250 μm, especially 50 to 150 μm.

The intermediate of the invention (i.e. the stabilised amorphous lenalidomide of the invention or the stabilised molecularly disperse lenalidomide of the invention) is usually employed to prepare a pharmaceutical formulation.

The subject matter of the invention is therefore a pharmaceutical formulation containing intermediate of the invention and pharmaceutical excipients.

These are the excipients with which the person skilled in the art is familiar, such as those which are described in the European Pharmacopoeia.

Examples of excipients used are disintegrants, anti-stick agents, emulsifiers, pseudo-emulsifiers, fillers, additives to improve the powder flowability, glidants, wetting agents, gelling agents and/or lubricants. Where appropriate, further excipients can also be used.

The ratio of active agent to excipients is preferably selected such that the resulting formulations contain
1 to 50% by weight, more preferably 2 to 25% by weight, in particular 5 to 15% by weight amorphous or molecularly disperse lenalidomide and
50 to 99% by weight, more preferably 75 to 98% by weight, especially 85 to 95% by weight pharmaceutically acceptable excipients.

In these ratios specified, the amount of surface stabiliser or matrix material optionally used in the preparation of the intermediate of the invention is counted as an excipient. This means that the amount of active agent refers to the amount of amorphous or molecularly disperse lenalidomide contained in the formulation.

It has been shown that the intermediates of the invention are suitable for serving both as a basis for a dosage form with immediate release (or “IR” for short) and also with modified release (or “MR” for short).

In a preferred embodiment for an IR formulation, a relatively large amount of disintegrant is used. In that preferred embodiment, the pharmaceutical formulation of the invention therefore contains
(i) 1 to 50% by weight, more preferably 2 to 25% by weight, especially 5 to 15% by weight amorphous or molecularly disperse lenalidomide and
(ii) 5 to 30% by weight, more preferably 10 to 25% by weight, especially 12 to 22% by weight disintegrants, based on the total weight the formulation.

“Disintegrants” is the term generally used for substances which accelerate the disintegration of a dosage form, especially a tablet, after it is placed in water. Suitable disintegrants are, for example, organic disintegrants such as carrageenan, croscarmellose, sodium carboxymethyl starch and crospovidone. Alkaline disintegrants are preferably used. The term “alkaline disintegrants” means disintegrants which, when dissolved in water, produce a pH level of more than 7.0.

More preferably, inorganic alkaline disintegrants are used, especially salts of alkali and alkaline earth metals. Preferred examples here are sodium, potassium, magnesium and calcium. As anions, carbonate, hydrogen carbonate, phosphate, hydrogen phosphate and dihydrogen phosphate are preferred. Examples are sodium hydrogen carbonate, sodium hydrogen phosphate, calcium hydrogen carbonate and the like.

Sodium hydrogen carbonate is particularly preferably used as a disintegrant, especially in the above-mentioned amounts.

In a preferred embodiment for an MR formulation, a relatively small amount of disintegrant is used. In that preferred embodiment, the pharmaceutical formulation of the invention therefore contains
(i) 1 to 50% by weight, more preferably 2 to 25% by weight, especially 5 to 15% by weight amorphous or molecularly disperse lenalidomide and
(ii) 0 to 10% by weight, more preferably 0.1 to less than 5% by weight, especially 1 to 4% by weight disintegrants, based on the total weight of the formulation.

In the case of the MR formulation, croscarmellose or crospovidone is preferred as the disintegrant.

In addition the conventional retardation techniques can be used for the MR formulation.

Furthermore, the pharmaceutical formulation (both for IR and for MR) preferably contains one or more of the above-mentioned excipients. These will be explained in more detail below.

The formulation of the invention preferably contains fillers. “Fillers” generally means substances which serve to form the body of the tablet in the case of tablets with small amounts of active agent (e.g. less than 70% by weight). This means that fillers “dilute” the active agents in order to produce an adequate tablet-compression mixture. The normal purpose of fillers, therefore, is to obtain a suitable tablet size. Fillers can likewise serve, in the case of a capsule or sachet formulation, to dilute the amount of active agent.

Examples of preferred fillers are lactose, lactose derivatives, starch, starch derivatives, treated starch, talcum, calcium phosphate, sucrose, calcium carbonate, magnesium carbonate, magnesium oxide, maltodextrin, calcium sulphate, dextrates, dextrin, dextrose, hydrogenated vegetable oil, kaolin, sodium chloride, and/or potassium chloride. Similarly, siliconated microcrystalline cellulose (Prosolv® Rettenmaier & Söhne, Germany) can be used.

Fillers are generally used in an amount of 1 to 80% by weight, preferably 10 to 70% by weight, more preferably 30 to 60% by weight, based on the total weight of the formulation.

The tablet of the invention may also contain additives to improve the powder flowability. One example of an additive to improve the powder flowability is disperse silica, e.g. known under the trade name Aerosil®. Preferably, silica is used with a specific surface area of 50 to 400 m²/g, determined by gas adsorption in accordance with Ph. Eur., 6th edition 2.9.26.

Additives to improve the powder flowability are generally used in an amount of 0.1 to 3% by weight, preferably 0.5 to 2.5% by weight, based on the total weight of the formulation.

In addition, lubricants may be used. Lubricants are generally used in order to reduce sliding friction. In particular, the intention is to reduce the sliding friction found during tablet pressing between the punches moving up and down in the die and the die wall, on the one hand, and between the edge of the tablet and the die wall, on the other hand. Suitable lubricants are, for example, stearic acid, adipic acid, sodium stearyl fumarate and/or magnesium stearate.

Lubricants are generally used in an amount of 0.1 to 3% by weight, based on the total weight of the formulation.

It lies in the nature of pharmaceutical excipients that they sometimes perform more than one function in a pharmaceutical formulation. In the context of this invention, in order to provide an unambiguous delimitation, the fiction will therefore preferably apply that a substance which is used as a particular excipient is not simultaneously also used as a further pharmaceutical excipient. Sorbitol, for example—if used as a surface stabiliser or matrix material—is not also used as a filler (even though sorbitol can also have a certain “diluting” effect).

The pharmaceutical formulation of the invention is preferably pressed into tablets. In the state of the art, wet granulation by means of a gelatine solution is proposed (see EP 0 925 294 B1, Example 20).

It has, however, become apparent that the properties of the resulting tablets can be improved if wet granulation is avoided.

The intermediates of the invention are therefore compressed into tablets by means of direct compression or are subjected to dry granulation before being compressed into tablets. Intermediates with a bulk density of less than 0.5 g/ml are preferably processed by dry granulation.

Direct compression is especially preferred if the intermediate is prepared by means of melt extrusion (process steps (a4) and (b4) or pellet layering (process steps (a2) and (b2)).

Dry granulation is preferred if the intermediate is prepared by means of spray-drying (process steps (a3) and (b3)), freeze-drying (process steps (al) and (b1)), melt-processing (process steps (a4) and (b4)) or milling (process steps (a5) and (b5)). In particular, it has unexpectedly been found that the preparation of the intermediate by means of spray-drying (process steps (a3) and (b3)) can advantageously be combined with dry granulation in order to solve the problems described at the beginning.

A further aspect of the present invention therefore relates to a dry-granulation method comprising the steps of
(I) preparing the intermediate of the invention and one or more pharmaceutical excipients (especially those described above);
(II) compacting it into flakes; and
(III) granulating or comminuting the flakes.

In step (I), the intermediate of the invention and excipients are preferably mixed. The mixing can be performed in conventional mixers. Alternatively, it is possible that the lenalidomide intermediate is initially only mixed with part of the excipients (e.g. 50 to 95%) before compacting (II), and that the remaining part of the excipients is added after the granulation step (III). In the case of multiple compacting, the excipients should preferably be mixed in before the first compacting step, between multiple compacting steps or after the last granulation step.

In step (II) of the method of the invention, the mixture from step (I) is compacted into flakes. It is preferable here that it should be dry compacting, i.e. the compacting is preferably performed in the absence of solvents, especially in the absence of organic solvents.

The compacting conditions are usually selected such that the intermediate of the invention is present in the form of compacted material (flakes), the density of the intermediate being 0.8 to 1.3 g/cm³, preferably 0.9 to 1.20 g/cm³, especially 1.01 to 1.15 g/cm³.

The term “density” here preferably relates to the “pure density” (i.e. not to the bulk density or tapped density). The pure density can be determined with a gas pycnometer. The gas pycnometer is preferably a helium pycnometer; in particular, the AccuPyc 1340 helium pycnometer from the manufacturer Micromeritics, Germany, is used.

The compacting is preferably carried out in a roll granulator.

The rolling force is usually 5 to 70 kN/cm, preferably 10 to 60 kN/cm, more preferably 15 to 50 kN/cm, especially 16 to 25 kN/cm.

The gap width of the roll granulator is, for example, 0.8 to 5 mm, preferably 1 to 4 mm, more preferably 1.5 to 3 mm, especially 1.8 to 2.8 mm.

The compacting apparatus used preferably has a cooling means. In particular, the cooling is such that the temperature of the compacted material does not exceed 50° C., especially 40° C.

In step (III) of the method, the flakes are granulated. The granulation can be performed with methods known in the state of the art.

In a preferred embodiment, the granulation conditions are selected such that the resulting particles (granules) have a volume-average particle size ((D50) value) of 50 to 800 μm, more preferably 100 to 750 μm, even more preferably 150 to 500 μm, especially 200 to 450 μm.

In a preferred embodiment, the granulation is performed in a screen mill. In this case, the mesh width of the screen insert is usually 0.1 to 5 mm, preferably 0.5 to 3 mm, more preferably 0.75 to 2 mm, especially 0.8 to 1.8 mm.

In a preferred embodiment, the method is adapted such that multiple compacting occurs, with the granules resulting from step (III) being returned one or more times to the compacting (II). The granules from step (III) are preferably returned 1 to 5 times, especially 2 to 3 times.

In addition, the granulation conditions are preferably selected such that the resulting granules have a bulk density of 0.3 to 0.85 g/ml, more preferably 0.4 to 0.8 g/ml, especially 0.5 to 0.7 g/ml. The Hausner factor is usually in the range from 1.02 to 1.3, more preferably from 1.03 to 1.25 and especially from 1.04 to 1.15. The “Hausner factor” in this context means the ratio of tapped density to bulk density. The bulk density and tapped density are determined in accordance with USP 24, test 616 “Bulk Density and Tapped Density”.

The granules resulting from step (III) can be further processed into pharmaceutical dosage forms. For this purpose, the granules are filled into sachets or capsules, for example. The granules resulting from step (III) are preferably pressed into tablets (IV).

In step (IV) of the method, the granules obtained in step (III) are pressed into tablets, i.e. the step involves compression into tablets. The compression can be performed with tableting machines known in the state of the art, such as eccentric presses or rotary presses. In the case of rotary presses, a compressive force of 2 to 40 kN, preferably 2.5 to 35 kN, is usually applied. As an example, the Fette® 102i press (Fette GmbH, Germany) is used.

In step (IV) of the method, pharmaceutical excipients may optionally be added to the granules from step (III).

The amounts of excipients added in step (IV) usually depend on the type of tablet to be produced and the amount of excipients which were already added in steps (I) or (II).

In the case of direct compression, only steps (I) and (IV) of the method described above are performed.

The tableting conditions are preferably selected such that the resulting tablets have a ratio of tablet height to weight of 0.005 to 0.3 mm/mg, particularly preferably 0.05 to 0.2 mm/mg.

In addition, the resulting tablets preferably have a hardness of 50 to 200 N, particularly preferably 80 to 150 N. The hardness is determined in accordance with Ph. Eur. 6.0, section 2.9.8.

In addition, the resulting tablets preferably have a friability of less than 5%, particularly preferably less than 3%, especially less than 2%. The friability is determined in accordance with Ph. Eur. 6.0, section 2.9.7.

Finally, the tablets of the invention usually have a “content uniformity” of 90 to 110% of the average content, preferably 95 to 105%, especially 98 to 102%. The “content uniformity” is determined in accordance with Ph. Eur.6.0, section 2.9.6.

In the case of an IR formulation, the release profile of the tablets of the invention according to the USP method (preferably paddle apparatus II, 900 ml 0.01 N HCl, pH 2, 37° C., 50 rpm) after 10 minutes usually indicates a content released of at least 30%, preferably at least 50%, especially at least 70%.

In the case of an MR formulation, the release profile of the tablets of the invention according to the USP method (preferably paddle apparatus II, 900 ml 0.01 N HCl, pH 2, 37° C., 50 rpm) after 60 minutes usually indicates a content released of 10%, preferably 20%, especially 30%.

The above details regarding hardness, friability, content uniformity and release profile preferably relate here to the non-film-coated tablet for an IR formulation. For a modified-release tablet, the release profile relates to the total formulation.

The tablets produced by the method of the invention may be tablets which can be swallowed unchewed (non-film-coated or preferably film-coated). They may likewise be chewable tablets or dispersible tablets. “Dispersible tablet” here means a tablet to be used for producing an aqueous suspension for swallowing.

In the case of tablets which are swallowed unchewed, it is preferable that they be coated with a film layer. For this purpose, the methods of film-coating tablets which are standard in the state of the art can be employed. The above-mentioned ratios of active agent to excipient, however, relate to the uncoated tablet.

For film-coating, macromolecular substances are preferably used, such as modified celluloses, polymethacrylates, polyvinyl pyrrolidone, polyvinyl acetate phthalate, zein and/or shellack or natural gum, such as carrageenan.

The thickness of the coating is preferably 1 to 100 μm, more preferably 2 to 80 μm.

The above explanations indicated the unexpectedly advantageous properties of non-crystalline lenalidomide (i.e. amorphous or molecularly disperse) lenalidomide. In a second aspect of the invention, an advantageous processing method will be explained, which solves the above-mentioned problems and which is especially suitable for use for the non-crystalline lenalidomide explained above, but also for crystalline lenalidomide.

It has unexpectedly been possible to solve the problems by means of the dry-processing of lenalidomide together with an adhesion promoter.

The subject matter of the second aspect of the invention is therefore a method of producing tablets containing lenalidomide and adhesion promoter, wherein the tablets are produced by means of dry granulation or direct compression. A further subject matter of the second aspect of the invention is tablets which are obtainable by means of the embodiments of the method of the invention described below.

Another subject matter of the second aspect of the invention is an intermediate, obtainable by jointly dry-compacting lenalidomide with an adhesion promoter.

In the context of the second aspect of this invention, the term “lenalidomide” comprises 3-(4-amino-1,3-dihydro-1-oxo-2H-isoindol-2-yl)-2,6-piperidine dione in accordance with formula (1) above. In addition, the term “lenalidomide” comprises all the pharmaceutically acceptable salts, hydrates and solvates thereof.

The salts may be acid addition salts. Examples of suitable salts are hydrochlorides, carbonates, hydrogen carbonates, acetates, lactates, butyrates, propionates, sulphates, hydrogen sulphates, methane sulphonates, citrates, tartrates, nitrates, sulphonates, oxalates and/or succinates.

Lenalidomide can be used in the context of the second aspect both in amorphous or molecularly disperse and also in crystalline form.

According to WO 2005/023192, crystalline lenalidomide may be present in eight different polymorphous forms (polymorphous forms A to H). In the context of this invention, it is preferable for the polymorphous forms A, B and/or E to be used. Polymorph B (hemihydrate) is particularly preferred.

The adhesion promoter is generally a substance which is suitable for stabilising lenalidomide in compacted or compressed form. The addition of the adhesion promoter usually leads to an increase in the size of the interparticulate surfaces, where bonds can form (e.g. during the compression process). In addition, adhesion promoters are characterised by the fact that they increase the plasticity of the tableting mixture, so that solid tablets form during compression.

In one possible embodiment, the adhesion promoter is a polymer. In addition, the term “adhesion promoter” also includes substances which behave like polymers. Examples of these are fats and waxes. Furthermore, the adhesion promoter also includes solid, non-polymeric compounds which preferably contain polar side groups. Examples of these are sugar alcohols or disaccharides. Finally, the term “adhesion promoter” also encompasses surfactants, especially surfactants which are present in solid form at room temperature.

The adhesion promoter used in the context of this invention is preferably a polymer which has a glass transition temperature (Tg) higher than 15° C., more preferably 40° C. to 150° C., especially 50° C. to 100° C.

The term “glass transition temperature” (Tg) is used to describe the temperature at which amorphous or partially crystalline polymers change from the solid state to the liquid state. In the process, a distinct change in physical parameters, e.g. hardness and elasticity, occurs. Below the Tg, a polymer is usually glassy and hard, whereas above the Tg, it changes into a rubber-like to viscous state. The glass transition temperature is determined in the context of this invention by means of dynamic differential scanning calorimetry (DSC). For this purpose a Mettler Toledo DSC 1 apparatus, for example, can be used. The work is performed at a heating rate of 1-20° C./min., preferably 5-15° C./min., and at a cooling rate of 5-25, preferably 10-20° C./min.

In addition, the polymer which can be used as an adhesion promoter preferably has a number-average molecular weight of 1,000 to 500,000 g/mol, more preferably 2,000 to 90,000 g/mol. When the polymer used in the preparation of the intermediate is dissolved in water in an amount of 2% by weight, the resulting solution preferably has a viscosity of 0.1 to 8 mPa/s, more preferably 0.3 to 7 mPa/s, especially 0.5 to 4 mPa/s, measured at 25° C.

Hydrophilic polymers are preferably used for the preparation of the intermediate. This refers to polymers which possess hydrophilic groups. Examples of suitable hydrophilic groups are hydroxy, alkoxy, acrylate, methacrylate, sulphonate, carboxylate and quaternary ammonium groups.

The intermediate of the invention may, for example, comprise the following polymers as adhesion promoters: polysaccharides, such as hydroxypropyl methyl cellulose (HPMC), carboxymethyl cellulose (CMC, especially sodium and calcium salts), ethyl cellulose, methyl cellulose, hydroxyethyl cellulose, ethyl hydroxyethyl cellulose, hydroxypropyl cellulose (HPC); microcrystalline cellulose, guar flour, alginic acid and/or alginates; synthetic polymers such as polyvinyl pyrrolidone, polyvinyl acetate (PVAC), polyvinyl alcohol (PVA), polymers of acrylic acid and their salts, polyacrylamide, polymethacrylates, vinyl pyrrolidone/vinyl acetate copolymers (such as Kollidon® VA64, BASF), polyalkylene glycols, such as polypropylene glycol or preferably polyethylene glycol, co-block polymers of polyethylene glycol, especially co-block polymers of polyethylene glycol and polypropylene glycol (Pluronic®, BASF), and mixtures of the polymers mentioned.

Substances particularly preferably used as adhesion promoters are polyvinyl pyrrolidone, preferably with a weight-average molecular weight of 10,000 to 60,000 g/mol, especially 12,000 to 40,000 g/mol, a copolymer of vinyl pyrrolidone and vinyl acetate, especially with a weight-average molecular weight of 40,000 to 70,000 g/mol and/or polyethylene glycol, especially with a weight-average molecular weight of 2,000 to 10,000 g/mol, and HPMC, especially with a weight-average molecular weight of 20,000 to 90,000 g/mol and/or preferably a content of methyl groups of 10 to 35% and a content of hydroxy groups of 1 to 35%. In addition, microcrystalline cellulose can preferably be used, especially one with a specific surface area of 0.7-1.4 m²/g. The specific surface area is determined by means of the gas adsorption method according to Brunauer, Emmet and Teller.

In addition, the adhesion promoter also includes solid, non-polymeric compounds which preferably contain polar side groups. Examples of these are sugar alcohols or disaccharides. Examples of suitable sugar alcohols and/or disaccharides are lactose, mannitol, sorbitol, xylitol, isomalt, glucose, fructose, maltose and mixtures thereof. The term “sugar alcohols” in this context also includes monosaccharides. Lactose and mannitol in particular are used as adhesion promoters.

Alternatively, it is possible to use waxes, such as cetyl palmitate or carnauba wax as adhesion promoters. It is likewise possible to use fats, such as glycerol fatty acid esters (e.g. glycerol palmitate, glycerol behenate, glycerol laurate, glycerol stearate) or PEG glycerol fatty acid esters.

Similarly, mixtures of the above-mentioned adhesion promoters are possible.

In preferred embodiments of the second aspect the present invention, lenalidomide and adhesion promoter are used in an amount in which the weight ratio of lenalidomide to adhesion promoter is 10:1 to 1:100, more preferably 1:1 to 1:75, even more preferably 1:2 to 1:50, especially 1:5 to 1:35.

It is advantageous for the adhesion promoter to be used in particulate form and for the volume-average particle size (D50) of the adhesion promoter to be less than 500 μm, preferably 5 to 200 μm.

The method according to the second aspect of the present invention can generally be carried out in two embodiments, namely as a dry-granulation process and as a direct-compression process. Both embodiments are carried out in the absence of solvent.

A first embodiment of the second aspect of the present invention therefore relates to a dry-granulation method comprising the steps of
(a) mixing lenalidomide with an adhesion promoter and optionally further pharmaceutical excipients;
(b) compacting it into flakes;
(c) granulating the flakes;
(d) compressing the resulting granules into tablets, optionally with the addition of further pharmaceutical excipients; and
(e) optionally film-coating the tablets.

In step (a), lenalidomide and adhesion promoter and optionally further pharmaceutical excipients (described below) are mixed. The mixing can be performed in conventional mixers. The mixing may, for example, be performed in compulsory mixers or free-fall mixers, e.g. using a Turbula T 10B (Bachofen AG, Switzerland). Alternatively, it is possible that the lenalidomide is initially only mixed with part of the excipients (e.g. 50 to 95%) before compacting (b), and that the remaining part of the excipients is added after the granulation step (c). In the case of multiple compacting, the excipients should preferably be mixed in before the first compacting step, between multiple compacting steps or after the final granulation step.

The mixing conditions in step (a) and/or the compacting conditions in step (b) are usually selected such that at least 30% of the surface of the resulting lenalidomide particles is covered with adhesion promoter, more preferably at least 50% of the surface, particularly preferably at least 70% of the surface, especially at least 90% of the surface.

In step (b) of the method of the invention, the mixture from step (a) is compacted into flakes. It is preferable here that it should be dry compacting, i.e. the compacting is preferably performed in the absence of solvents, especially in the absence of organic solvents.

The compacting is preferably carried out in a roll granulator.

The rolling force is preferably 5 to 70 kN/cm, preferably 10 to 60 kN/cm, more preferably 15 to 50 kN/cm.

The gap width of the roll granulator is, for example, 0.8 to 5 mm, preferably 1 to 4 mm, more preferably 1.5 to 3 mm, especially 1.8 to 2.8 mm.

The compacting apparatus used preferably has a cooling means. In particular, the cooling is such that the temperature of the compacted material does not exceed 50° C., especially 40° C.

In step (c) of the method, the flakes are granulated. The granulation can be performed with methods known in the state of the art. A Comill® U5 apparatus (Quadro Engineering, USA), for example, is used for granulating.

In a preferred embodiment, the granulation conditions are selected such that the resulting particles (granules) have a volume-average particle size ((D50) value) of 50 to 800 μm, more preferably 100 to 750 μm, even more preferably 150 to 500 μm, especially 200 to 450 μm.

In addition, the granulation conditions can be selected such that no more than 55% of the particles are less than 200 μm in size or that the average particle diameter (D50) is between 100 and 450 μm.

In addition, the granulation conditions are preferably selected such that the resulting granules have a bulk density of 0.2 to 0.85 g/ml, more preferably 0.3 to 0.8 g/ml, especially 0.4 to 0.7 g/ml. The Hausner factor is usually in the range from 1.03 to 1.3, more preferably from 1.04 to 1.20 and especially from 1.04 to 1.15. The “Hausner factor” in this context means the ratio of tapped density to bulk density.

In a preferred embodiment, the granulation is performed in a screen mill. In this case, the mesh width of the screen insert is usually 0.1 to 5 mm, preferably 0.5 to 3 mm, more preferably 0.75 to 2 mm, especially 0.8 to 1.8 mm.

In a preferred embodiment, the method is adapted such that multiple compacting occurs, with the granules resulting from step (c) being returned one or more times to the compacting (b). The granules from step (c) are preferably returned 1 to 5 times, especially 2 to 3 times.

The granules resulting from step (c) can be further processed into pharmaceutical dosage forms. For this purpose, the granules are filled into sachets or capsules, for example. A subject matter of the invention is therefore also capsules and sachets containing a granulated pharmaceutical composition which is obtainable by the dry-granulation process of the invention.

The granules resulting from step (c) are preferably pressed into tablets (=step (d) of the method of the invention).

In step (d), compression into tablets occurs. Compression can be performed with tableting machines known in the state of the art. The compression is preferably performed in the absence of solvents.

Examples of suitable tableting machines are eccentric presses or rotary presses. As an example, the Fette® 102i press (Fette GmbH, DE) can be used. In the case of rotary presses, a compressive force of 2 to 40 kN, preferably 2.5 to 35 kN, is usually applied.

In step (d) of the method, pharmaceutical excipients may optionally be added to the granules from step (c). The amounts of excipients added in step (d) usually depend on the type of tablet to be produced and the amount of excipients which were already added in steps (a) or (b).

In the optional step (e) of the method of the invention, the tablets from step (d) are film-coated. For this purpose, the methods of film-coating tablets which are standard in the state of the art can be employed.

For film-coating, macromolecular substances are preferably used, such as modified celluloses, polymethacrylates, polyvinyl pyrrolidone, polyvinyl acetate phthalate, zein and/or shellack or natural gum, such as carrageenan.

The thickness of the coating is preferably 1 to 100 μm.

In addition to the dry-compacting and granulation processes described above, another point of the second aspect of the present invention is a compacted intermediate containing lenalidomide. Another subject matter of the second aspect of the invention is therefore an intermediate, obtainable by jointly dry-compacting lenalidomide with an adhesion promoter.

As regards the properties of the lenalidomide to be used and the adhesion promoter to be used, reference may be made to the above explanations. The intermediate of the invention can be produced by steps (a) and (b) of the method of the invention explained above.

The compacting conditions for preparing the intermediate of the invention are usually selected such that the intermediate of the invention is present in the form of compacted material (flakes), the density of the intermediate being 0.8 to 1.3 g/cm³, preferably 0.9 to 1.20 g/cm³, especially 1.01 to 1.15 g/cm³.

The term “density” here preferably relates to the “pure density” (i.e. not to the bulk density or tapped density). The pure density can be determined with a gas pycnometer. The gas pycnometer is preferably a helium pycnometer; in particular, the AccuPyc 1340 helium pycnometer from the manufacturer Micromeritics, Germany, is used.

It is preferable that that type and quantity of the adhesion promoter should be selected such that the resulting intermediate has a glass transition temperature (Tg) of more than 20° C., preferably >30° C.

It is preferable that the type and quantity of the adhesion promoter should be selected such that the resulting intermediate is storage-stable. “Storage-stable” means that in the intermediate of the invention, after storage for 3 years at 25° C. and 50% relative humidity, the proportion of crystalline lenalidomide—based on the total amount of lenalidomide—is no more than 60% by weight, preferably no more than 30% by weight, more preferably no more than 15% by weight, in particular no more than 5% by weight.

All the above remarks on the intermediate of the invention also apply to the product of the process resulting in step (b).

As described above under step (c) of the method of the invention, the intermediates of the invention may be comminuted, e.g. granulated. Normally, the intermediates of the invention are present in particulate form and have an average particle diameter (D50) of 1 to 750 μm, preferably von 1 to 350 μm, depending on the preparation method in each case.

The expression “average particle diameter” always relates in the context of this invention to the D50 value of the volume-average particle diameter determined by means of laser diffractometry. In particular, a Malvern Instruments Mastersizer 2000 was used to determine the diameter (wet measurement with ultrasound for 60 sec., 2,000 rpm, the evaluation being performed using the Fraunhofer model), and preferably using a dispersant in which the substance to be measured does not dissolve at 20° C.). The average particle diameter, which is also referred to as the D50 value of the integral volume distribution, is defined in the context of this invention as the particle diameter at which 50% by volume of the particles have a smaller diameter than the diameter which corresponds to the D50 value. Similarly, 50% by volume of the particles then have a larger diameter than the D50 value. The terms “average particle size” and “average particle diameter” are used synonymously in the context of this application.

The intermediate of the invention is usually employed to prepare a pharmaceutical formulation. For this purpose, the intermediate—together with further excipients where applicable—is filled into sachets or capsules, for example. As described above under step (d) of the method of the invention, the intermediate of the invention is preferably compressed into tablets.

In the case of direct compression, only steps (a) and (d) and, where applicable, (e) of the method described above are performed. One subject matter of the second aspect of the invention is therefore a method comprising the steps of
(a) mixing lenalidomide with an adhesion promoter and optionally further pharmaceutical excipients; and
(d) directly compressing the resulting mixture into tablets, and then
(e) optionally film-coating the tablets.

In principle, the explanations provided above on steps (a), (d) and (e) also apply to direct compression.

In a preferred embodiment, in the case of direct compression, step (a) includes jointly milling lenalidomide and adhesion promoter. Where appropriate, further pharmaceutical excipients can be added.

The mixing conditions are usually selected such that at least 30% of the surface of the resulting lenalidomide particles is covered with adhesion promoter, more preferably at least 50% of the surface, particularly preferably at least 70% of the surface, especially at least 90% of the surface.

The milling is generally performed in conventional milling apparatuses, such as in a ball mill, air jet mill, pin mill, classifier mill, cross beater mill, disk mill, mortar grinder, rotor mill. The milling time is usually 0.5 minutes to 1 hour, preferably 2 minutes to 50 minutes, more preferably 5 minutes to 30 minutes.

In the case of direct compression, it is preferable that in step (d), a mixture is used in which the particle sizes of the active agent and the excipients are matched to one another. Preferably, lenalidomide, adhesion promoter and, where applicable, any further pharmaceutical excipients are used in particulate form with an average particle size (D50) of 35 to 250 μm, more preferably 50 to 200 μm, especially 70 to 150 μm.

Both in the case of dry granulation and in the case of direct compression, further pharmaceutical excipients may be used in addition to lenalidomide and adhesion promoter. These are the excipients with which the person skilled in the art is familiar, especially those which are described in the European Pharmacopoeia.

Examples of excipients used are disintegrants, anti-stick agents, emulsifiers, pseudo-emulsifiers, fillers, additives to improve the powder flowability, glidants, wetting agents, gelling agents and/or lubricants. Where appropriate, further excipients can also be used.

“Disintegrants” is the term generally used for substances which accelerate the disintegration of a dosage form, especially a tablet, after it is placed in water. Suitable disintegrants are, for example, organic disintegrants such as carrageenan, croscarmellose and crospovidone. Alkaline disintegrants are likewise used. The term “alkaline disintegrants” means disintegrants which, when dissolved in water, produce a pH level of more than 7.0.

Inorganic alkaline disintegrants are preferably used, especially salts of alkali and alkaline earth metals. Preferred examples here are sodium, potassium, magnesium and calcium. As anions, carbonate, hydrogen carbonate, phosphate, hydrogen phosphate and dihydrogen phosphate are preferred. Examples are sodium hydrogen carbonate, sodium hydrogen phosphate, calcium hydrogen carbonate and the like.

The formulation of the invention usually contains fillers. “Fillers” generally means substances which serve to form the body of the tablet in the case of tablets with small amounts of active agent (e.g. less than 70% by weight). This means that fillers “dilute” the active agents in order to produce an adequate tablet-compression mixture. The normal purpose of fillers, therefore, is to obtain a suitable tablet size.

Examples of preferred fillers are lactose, lactose derivatives, starch, starch derivatives, treated starch, talcum, calcium phosphate, sucrose, calcium carbonate, magnesium carbonate, magnesium oxide, maltodextrin, calcium sulphate, dextrates, dextrin, dextrose, hydrogenated vegetable oil, kaolin, sodium chloride, and/or potassium chloride. Prosolv® (Rettenmaier & Söhne, Germany) can likewise be used.

Fillers are normally used in an amount of 1 to 80% by weight, more preferably 20 to 60% by weight, based on the total weight of the formulation.

One example of an additive to improve the powder flowability is disperse silica, e.g. known under the trade name Aerosil®.

Additives to improve the powder flowability are generally used in an amount of 0.1 to 3% by weight, based on the total weight of the formulation.

In addition, lubricants may be used. Lubricants are generally used in order to reduce sliding friction. In particular, the intention is to reduce the sliding friction found during tablet pressing between the punches moving up and down in the die and the die wall, on the one hand, and between the edge of the tablet and the die wall, on the other hand. Suitable lubricants are, for example, stearic acid, adipic acid, sodium stearyl fumarate and/or magnesium stearate.

Lubricants are generally used in an amount of 0.1 to 3% by weight, based on the total weight of the formulation.

The ratio of active agent to excipients is preferably selected such that the formulations resulting from the method of the invention (i.e. the tablets of the invention for example) contain
1 to 50% by weight, more preferably 2 to 25% by weight, especially 5 to 15% by weight lenalidomide and
50 to 99% by weight, more preferably 75 to 98% by weight, especially 85 to 95% by weight pharmaceutically acceptable excipients.

In these ratios specified, the amount of adhesion promoter used in the method of the invention or used to prepare the intermediate of the invention is counted as an excipient. This means that the amount of active agent refers to the amount of lenalidomide contained in the formulation.

It has been shown that the formulations of the second aspect of the invention (i.e. the tablets of the invention or the granules of the invention which result from step (c) of the second aspect of the method of the invention and which can be filled into capsules or sachets, for example) may serve both as a dosage form with immediate release (or “IR” for short) and also with modified release (or “MR” for short).

In a preferred embodiment for an IR formulation, a relatively large amount of disintegrant is used. In that preferred embodiment, the pharmaceutical formulation of the invention therefore contains
(i) 1 to 50% by weight, more preferably 2 to 25% by weight, especially 5 to 15% by weight lenalidomide and
(ii) 2 to 30% by weight, more preferably 5 to 25% by weight, especially 12 to 22% by weight disintegrants, based on the total weight the formulation.

In a preferred embodiment for an MR formulation, a relatively small amount of disintegrant is used. In that preferred embodiment, the pharmaceutical formulation of the invention therefore contains
(i) 1 to 50% by weight, more preferably 2 to 25% by weight, especially 5 to 15% by weight lenalidomide and
(ii) 0 to 10% by weight, more preferably 0.1 to less than 5% by weight, especially 1 to 4% by weight disintegrants, based on the total weight of the formulation.

In the case of the MR formulation, croscarmellose or crospovidone is preferred as the disintegrant. In the case of the IR formulation, alkaline disintegrants are preferred.

In addition the conventional retardation techniques can be used for the MR formulation.

The above-mentioned pharmaceutical excipients can be used in both the preferred embodiments (dry granulation and direct compression). Furthermore, the tableting conditions in both embodiments of the method of the invention are preferably selected such that the resulting tablets have a ratio of tablet height to weight of 0.005 to 0.3 mm/mg, particularly preferably 0.05 to 0.2 mm/mg.

In addition, the resulting tablets preferably have a hardness of 50 to 200 N, particularly preferably 80 to 150 N. The hardness is determined in accordance with Ph. Eur. 6.0, section 2.9.8.

In addition, the resulting tablets preferably have a friability of less than 5%, particularly preferably less than 3%, especially less than 2%. The friability is determined in accordance with Ph. Eur. 6.0, section 2.9.7.

Finally, the tablets of the invention usually have a “content uniformity” of 90 to 110% of the average content, preferably 95 to 105%, especially 98 to 102%. The “content uniformity” is determined in accordance with Ph. Eur. 6.0, section 2.9.6.

In the case of an IR formulation, the release profile of the tablets of the invention after 10 minutes according to the USP method usually indicates a content released of at least 30%, preferably at least 50%, especially at least 70%.

In the case of an MR formulation, the release profile of the tablets of the invention after 60 minutes according to the USP method usually indicates a content released of 10%, preferably 20%, especially 30%.

The above details regarding hardness, friability, content uniformity and release profile preferably relate here to the non-film-coated tablet for an IR formulation. For a modified-release tablet, the release profile relates to the total formulation.

The tablets produced by the method of the second aspect of the invention may be tablets which can be swallowed unchewed (non-film-coated or preferably film-coated). They may likewise be dispersible tablets. “Dispersible tablet” here means a tablet to be used for producing an aqueous suspension for swallowing.

In the case of tablets which are swallowed unchewed, it is preferable that they be coated with a film layer. For this purpose, the methods of film-coating tablets which are standard in the state of the art can be employed. The above-mentioned ratios of active agent to excipient, however, relate to the uncoated tablet.

As explained above, the subject matter of the second aspect of the invention is not only the method of the invention, but also the tablets produced with that method. It has further been found that the tablets produced with this method preferably have a bimodal pore size distribution. Hence, the subject matter the invention comprises tablets containing lenalidomide or a pharmaceutically acceptable salt thereof and adhesion promoter and optionally pharmaceutically acceptable excipients, wherein the tablets have a bi-modal pore size distribution.

This tablet of the invention is formed when the granules from process step (c) are compressed. This compressed material consists of solid material and pores. The pore structure can be characterised more specifically by determining the pore size distribution.

The pore size distribution was determined by means of mercury porosimetry. Mercury porosimetry measurements were made with the Micromeritics, Norcross, USA, “Poresizer” porosimeter. The pore sizes were calculated assuming a mercury surface tension of 485 mN/m. The cumulative pore volume was used to calculate the pore size distribution as the cumulative frequency distribution or proportion of the pore fractions in percent. The average pore diameter (4V/A) was determined from the total specific mercury intrusion volume (Vgesᵢₙₜ) and the total pore surface area (Agespₚₒᵣ) according to the following equation.

$$4 V / A = \frac{4 \cdot {Vges}_{int} [ ml \text{/} g ]}{{Ages}_{por} [ {m}^{2} \text{/} g ]}$$

“Bimodal pore size distribution” is understood to mean that the pore size distribution has two maxima. The two maxima are not necessarily separated by a minimum, but rather a head and shoulders pattern is also regarded as bimodal for the purposes of the invention.

The second aspect of the present invention can be summed up by the following points:
1. A method of producing tablets containing lenalidomide and adhesion promoter, wherein the tablets are produced by dry granulation or by direct compression.
2. The method according to point 1, comprising the steps of
   (a) mixing lenalidomide with an adhesion promoter and optionally further pharmaceutical excipients;
   (b) compacting it into flakes;
   (c) granulating the flakes;
   (d) compressing the resulting granules into tablets, optionally with the addition of further pharmaceutical excipients; and
   (e) optionally film-coating the tablets.
3. The method according to point 2, wherein the compacting (b) is performed in a roll compacter and the rolling force is 5 to 70 kN/cm, preferably 10 to 50 kN/cm.
4. The method according to points 2 or 3, wherein the granulation conditions in step (c) are selected such that no more than 55% of the particles are less than 200 μm in size or that the average particle diameter (D50) is between 100 and 450 μm.
5. The method according to point 1, comprising the steps of
   (a) mixing lenalidomide with an adhesion promoter and optionally further pharmaceutical excipients; and
   (d) directly compressing the resulting mixture into tablets, and then
   (e) optionally film-coating the tablets.
6. The method according to point 5, wherein step (a) includes jointly milling lenalidomide and adhesion promoter.
7. The method according to points 5 or 6, wherein in step (d), a mixture of lenalidomide, adhesion promoter and optionally further pharmaceutical excipients with an average particle size (D50) of 50 to 250 μm is used.
8. A tablet obtainable by a method according to any of points 1 to 7.
9. A tablet containing lenalidomide and adhesion promoter, wherein the tablet has a bimodal pore size distribution.
10. An intermediate obtainable by jointly dry-compacting lenalidomide with an adhesion promoter.
11. The intermediate according to point 10, wherein the density of the intermediate is 0.8 to 1.3 g/cm³, preferably 0.9 to 1.20 g/cm³.
12. The intermediate according to points 10 or 11, wherein the adhesion promoter used is a polymer with a weight-average molecular weight of less than 90,000 g/mol and a glass transition temperature (Tg) of more than 20° C. after being heated up twice.
13. The intermediate according to any of points 10 to 12, wherein a sugar alcohol is used as the adhesion promoter.
14. The intermediate according to any of points 10 to 13, wherein the weight ratio of lenalidomide to adhesion promoter is 5:1 to 1:75.
15. Tablets according to points 8 or 9 with a friability of less than 3%, a content uniformity of 95 to 105% and a hardness of 50 to 150 N.

The invention will now be illustrated with reference to the following examples.

### EXAMPLES

### Example Series I: Amorphous Lenalidomide

### Example I-1a

### Preparation of the Intermediate by Milling

The following batch for 1,000 dosage forms was produced.

5 g lenalidomide were milled for 10 h with 5 g HPMC and 0.3 g Aerosil in a ball mill.

It was possible to carry out the further processing in accordance with Examples I-6 and I-7.

### Example I-1b

### Preparation of the Intermediate by Milling

The following formulation for 1,000 dosage forms was milled as described under Example I-1a:

5 g lenalidomide

5 g Povidon® 25

2 g Aerosil®

### Example I-1c

### Preparation of the Intermediate by Milling

The following formulation for 1,000 dosage forms was milled as described under Example I-1a:

5 g lenalidomide

5 g isomalt

1 g L-HPC

### Example I-2

### Preparation of the Intermediate by Lyophilisation

The following batch for 1,000 dosage forms was produced.

5 g lenalidomide were dissolved in water/ethanol together with 10 g mannitol. That solution was reduced in temperature to −55° C. and frozen. Once the conductivity had reached less than 2%, the frozen mixture, at a temperature determined by the point of intersection between the product temperature and Rx −10° C. was dried at a pressure of less than 0.1 mbar, or the solvent was removed by sublimation.

After drying, the lyophilised material was heated to room temperature (20-25° C.).

It was possible to carry out the further processing in accordance with Examples I-6 and I-7.

### Example I-3a

### Preparation of the Intermediate by Melt Extrusion

The following batch for 1,000 dosage forms was produced.

5 g lenalidomide were extruded in a Leistritz micro 1 melt extruder together with 10 g PEG 8000 and 1 g Pluronic® F68 with a temperature cascade of 80-180° C. The strands of extruded material were cooled.

### Example I-3b

### Preparation of the Intermediate by Melt Extrusion

The following batch for 1,000 dosage forms was produced.

5 g lenalidomide were extruded in a Leistritz micro 1 melt extruder together with 10 g Povidon® VA64 and with a temperature cascade of 80-180° C. The strands of extruded material were cooled.

After screening, it was possible to carry out the further processing in accordance with Examples I-6 and I-7.

### Example I-4a

### Preparation of the Intermediate by Pellet Layering

The following batch for 1,000 dosage forms was produced.

5 g lenalidomide were dissolved in water/ethanol together with 20 g Povidon® VA 64 and applied to 200 g Cellets®.

During the process, the air inlet temperature was approx. 60-80° C., the product temperature 32-40° C. and the spray pressure approx. 1-1.5 bar.

It was possible to carry out the further processing in accordance with Examples I-6 and I-7.

### Example I-4b

### Preparation of the Intermediate by Pellet Layering

The pellet layering was carried out as described, the following batch being used:

5 g lenalidomide

12 g sorbitol

1.5 g talcum

### Example I-5a

### Preparation of the Intermediate by Spray-Drying

The following batch for 1,000 dosage forms was produced.

5 g lenalidomide were dissolved in water/ethanol together with 10 g HPMC and 2 g citric acid and spray-dried on a Büchi TYP B 191 spray tower The following parameters were maintained in the process:

Temperature 130° C., spray rate 5-20%, aspirator power 35-90%, flow control 30-75%.

The spray-dried material underwent a final drying stage for 24 h at 30° C. in a tray drying cabinet.

It was possible to carry out the further processing in accordance with Examples I-6 and I-7.

### Example I-5b

### Preparation of the Intermediate by Spray-Drying

The spray-drying was carried out as described in Example I-5a, the following batch being used:

5 g lenalidomide

5 g Avicel® PH 102

3 g Povidon® 25

### Example I-5c

### Preparation of the Intermediate by Spray-Drying

The spray-drying was carried out as described in Example I-5a, the following batch being used:

5 g lenalidomide

10 g Povidon® VA 64

### Example I-5d

### Preparation of the Intermediate by Spray-Drying

The spray-drying was carried out as described in Example I-5a, the following batch being used:

5 g lenalidomide

10 g HPMC

### Example I-6

### Production of Tablets

In order to produce tablets, the following formulation was used.

| | | |
|---|---|---|
| 1. Intermediate according to Example I-5d | 15 | mg |
| 2. Prosolv ® | 110 | mg |
| 3. Talcum | 1 | mg |
| 4. Sodium bicarbonate | 25 | mg |
| 5. Magnesium stearate | 1.5 | mg |
| 6. Aerosil ® | 0.8 | mg |

Ingredients 1 and 3 were pre-mixed for 5 min. in a free-fall mixer (Turbula TB 10). This mixture was compacted with 70% of the ingredients 2, 4, 5 and 6 using a roll compactor and screened with a mesh width of 1.25 mm. The compacted material was mixed with the remaining substances and pressed into tablets.

Example I-7

### Production of Capsules

In order to produce capsules, the following formulation was used.

| | | |
|---|---|---|
| 1. Intermediate according to Example I-5d | 15 | mg |
| 2. Lactose monohydrate | 80 | mg |
| 3. Microcrystalline cellulose | 60 | mg |
| 4. Talcum | 1 | mg |
| 5. Sodium bicarbonate | 15 | mg |
| 6. Magnesium stearate | 1.5 | mg |
| 7. Aerosil ® | 0.8 | mg |

Ingredients 1 and 4 were pre-mixed for 5 min. in a free-fall mixer (Turbula® TB 10). This mixture was compacted with 70% of the remaining ingredients using a roll compactor and screened with a mesh width of 1.25 mm. The compacted material was mixed with the remaining substances and filled into capsules.

### Example I-8

### Preparation of the Intermediate in the Melt

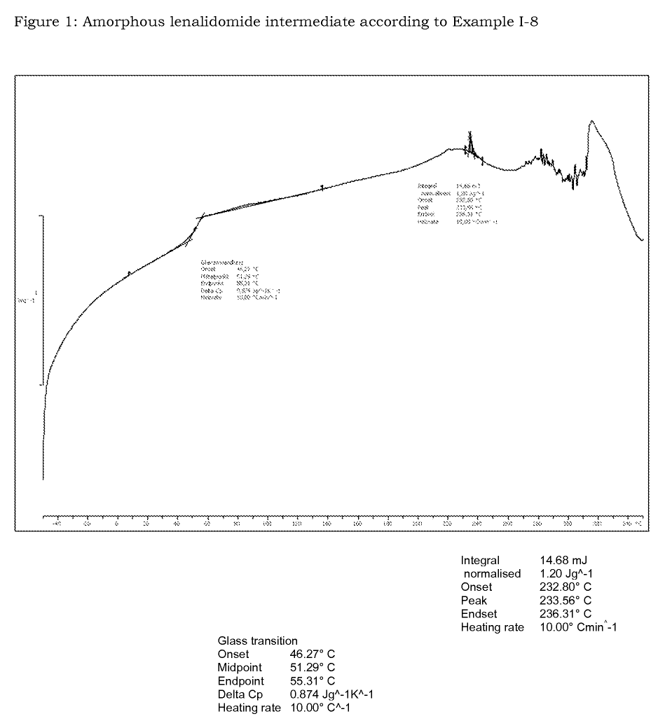

1 g lenalidomide was dissolved in 3 g molten isomalt. The melt was cooled, comminuted in a mortar and then passed through a screen with a mesh width of 630 μM. A DSC of the resulting amorphous lenalidomide intermediate is shown in FIG. 1.

### Example I-9

### Preparation of the Intermediate in the Melt

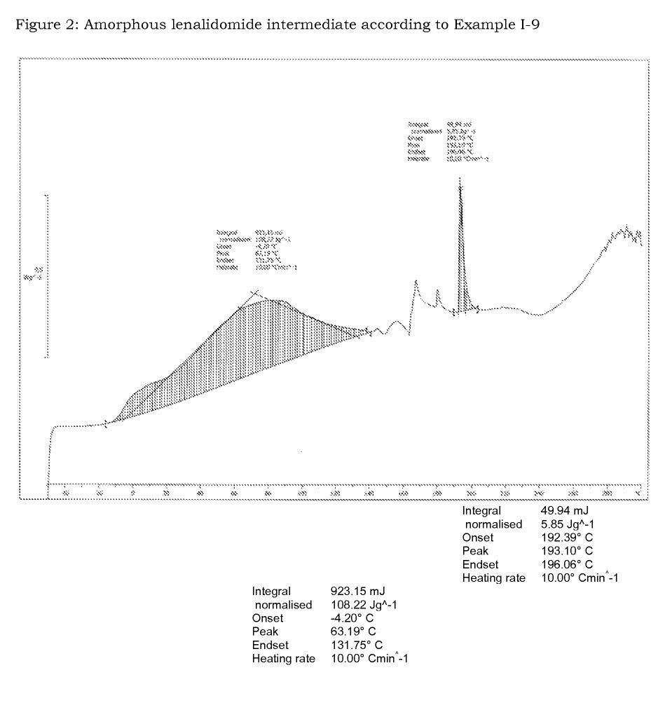

0.1 g lenalidomide was dissolved in 0.5 g molten isomalt. The melt was cooled, comminuted in a mortar and then passed through a screen with a mesh width of 630 μM. A DSC of the resulting amorphous lenalidomide intermediate is shown in FIG. 2.

### Example I-10

### Preparation of the Intermediate in the Melt

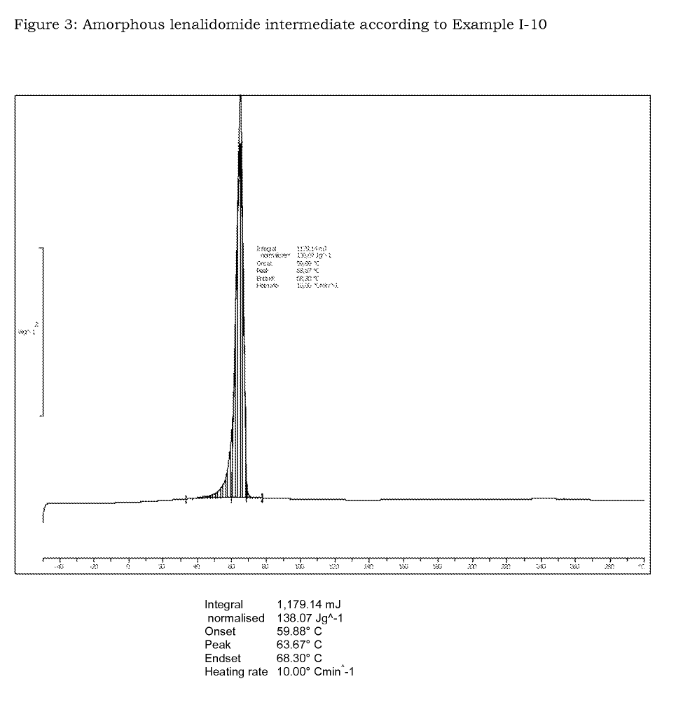

0.5 g lenalidomide was dissolved together with 5 g PEG 8000. The melt was cooled, comminuted in a mortar and then passed through a screen with a mesh width of 630 μM. A DSC of the resulting amorphous lenalidomide intermediate is shown in FIG. 3.

Example I-11

### Production of Tablets and Capsules

a) In order to produce tablets and capsules, the following formulation was used.

| | | |
|---|---|---|
| 1. Lenalidomide | 5.00 | mg |
| 2. Isomalt | 15.00 | mg |
| 3. Lactose monohydrate (Tablettose 70) | 50.00 | mg |
| 4. Microcrystalline cellulose (Avicel PH 102) | 55.00 | mg |
| 5. Carboxymethyl cellulose Na | 10.00 | mg |
| 6. Aerosil ® | 0.50 | mg |
| 7. Magnesium stearate | 1.00 | mg |

Ingredients 3, 4, 5 and 6 were passed through a screen with a mesh width of 630 μm and then pre-mixed for 10 min. in a free-fall mixer (Turbula® TB 10). To this mixture was added a melt prepared from 1 and 2 according to Example I-8 and mixed for a further 5 min. After that, screened magnesium stearate (screen with a mesh width of 250 μm) was added to the mixture and mixed for a further 3 min.

b) Production of Capsules

The mixture prepared in a) was filled into size 2 capsules.

c) Production of Tablets by Direct Compression

The mixture prepared in a) was compressed directly into tablets.

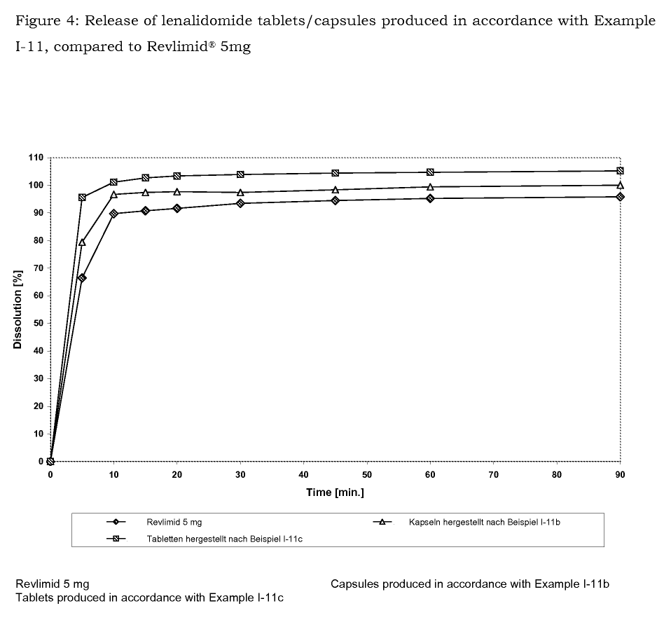

The in vitro release of the dosage forms of the invention in accordance with Example I-11 is compared in FIG. 4 with the commercially available dosage form Revlimid®. Measuring conditions: Paddle apparatus II USP:900 mL 0.01N HCl pH 2-37° C.-50 rpm.

### Example Series II: Lenalidomide in the Form of a Solid Solution

### Example II-1

### Preparation of the Intermediate by Melt Extrusion and Subsequent Compression into Tablets

The active agent was melted with Povidon® VA 64 in a ratio of 1:10 in the melt extruder at temperatures of less than 200° C. and extruded in a temperature cascade. A die plate with a hole diameter of 1 mm was used. The Leistritz® micro 18 twin-screw extruder was equipped with various screw elements. A kneading unit was installed in order to ensure the necessary thorough mixing and dissolution of the active agent in the polymer.

The cooled extruded material obtained was screened to 1.00 mm on a Comill® U5.

After that, it was premixed with talcum, then mixed with Avicel®, Lactose, sodium bicarbonate, Aerosil® and magnesium stearate (Turbula® T10B) and compressed into a tablet (Fette® 102 i). That tablet was coated with HPMC in a Pan-coater, e.g. Lödige® LHC 25. The coating solution also contained colorant, PEG, talcum and titanium dioxide.

| | | |
|---|---|---|
| Lenalidomide | 15 | mg |
| Povidon ® VA 64 | 150 | mg |
| Talcum | 6 | mg |
| Avicel ® | 30 | mg |
| Lactose | 20 | mg |
| Aerosil ® | 1.3 | mg |
| Magnesium stearate | 2.6 | mg |
| Sodium bicarbonate | 20.64 | mg |
| HPMC | 3 | mg |
| PEG | 0.5 | mg |
| Talcum | 1 | mg |
| Titanium dioxide | 0.4 | mg |
| Colorant | 0.1 | mg |

### Example II-2

### Preparation of the Intermediate by Pellet Layering and Filling into Capsules

The active agent was dissolved with sorbitol in ethanol/water and applied to a neutral pellet in the fluidised bed apparatus (GPC3 Glatt). During the process, the air inlet temperature was approx. 65° C., the spray pressure approx. 1 bar, and the nozzle size 1.2 mm. During the process, intervals were arranged. The cooled pellets were filled into capsules.

| | |
|---|---|
| Lenalidomide | 25 mg |
| Sorbitol | 160 mg |
| Sugarspheres ® | 200 mg |

### Example II-3

### Preparation of the Intermediate by Spray-Drying and Subsequent Compression into Tablets

The active agent was dissolved in water with HPMC and citric acid. That solution was spray-dried in a Büchi Mini-Spray-Dryer.

The spray-dried material was premixed with Lutrol, compacted with sodium bicarbonate and Pruv® and Prosolv®, and compressed into a tablet with the remaining amount of excipients. That tablet was coated with HPMC in a pan-coater, e.g. Lödige® LHC 25. The coating solution also contained colorant, PEG, talcum and titanium dioxide.

| | | |
|---|---|---|
| Lenalidomide | 25 | mg |
| HPMC | 160 | mg |
| Citric acid | 20.04 | mg |
| Lutrol ® (polyethylene glycol 400) | 2 | mg |
| Prosolv ® | 80 | mg |
| Sodium bicarbonate | 20 | mg |
| Sodium stearyl fumarate | 2.6 | mg |
| HPMC | 3 | mg |
| PEG | 0.5 | mg |
| Talcum | 1 | mg |
| Titanium dioxide | 0.4 | mg |
| Colorant | 0.1 | mg |

### Example II-4

### Preparation of the Intermediate by Spray-Drying and Filling into Capsules

a) Preparation of the Intermediate

| | |
|---|---|
| Lenalidomide | 10 g |
| Acetone | 700 g |
| EtOH 99% | 150 g |
| Kollidon ® VA 64 | 70 g |

Kollidon® VA 64 and lenalidomide were dissolved in acetone/EtOH. That solution was spray-dried in a Büchi Mini-Spray-Dryer.

b) Preparation of the Compacted Material and Filling into Capsules

| | | |
|---|---|---|
| Lenalidomide | 5.00 | mg |
| Kollidon VA 64 | 35.00 | mg |
| Lactose monohydrate (Tablettose ® 100) | 50.00 | mg |
| Microcrystalline cellulose | 55.00 | mg |
| Silica (Aerosil ® 300) | 0.50 | mg |
| Croscarmellose sodium | 25.00 | mg |
| Sodium stearyl fumarate | 1.0 | mg |

A mixture of lactose monohydrate, microcrystalline cellulose, Aerosil and croscarmellose sodium was passed through a screen with a mesh width of 630 μm and then pre-mixed for 10 min. in a free-fall mixer (Turbula TB 10). To that mixture was added a spray-dried mixture of lenalidomide and Kollidon® VA 64, produced in accordance with Example II-4a. The complete mixture s was passed through a screen with a mesh width of 500 μm and mixed for a further 5 min. After that, sodium stearyl fumarate was added to that mixture and mixed for a further 3 min.

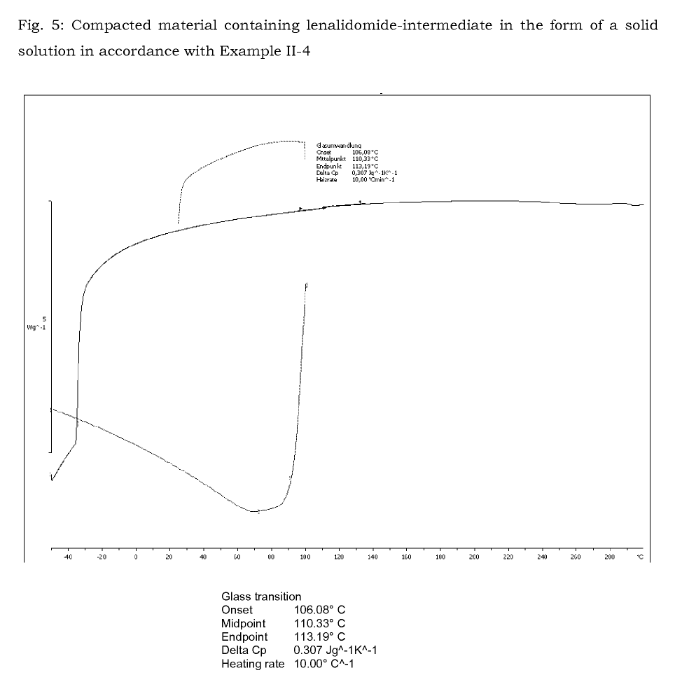

The resulting mixture was compacted using a roll compactor and screened on a Comill® U5 with a mesh width of 1.00 mm. A DSC of the compacted material containing lenalidomide intermediate in the form of a solid solution is illustrated in FIG. 5.

The compacted material was filled into size 2 capsules.

### Example II-5

### Preparation of the Intermediate in the Melt

1 g lenalidomide was dissolved in 10 g molten isomalt. The melt was cooled, comminuted in a mortar and then passed through a screen with a mesh width of 630 μM.

### Example Series III: Dry-Processing Lenalidomide

### Example III-1

### Direct Compression of Crystalline Lenalidomide

| | | |
|---|---|---|
| Lenalidomide (form B) | 5 | mg |
| Lactose monohydrate (Tablettose; Meggle) | 50 | mg |
| MCC (Avicel ® PH 102) | 55 | mg |
| Magnesium stearate | 1 | mg |
| Silica (Aerosil ® 300) | 0.5 | mg |
| Croscarmellose (Acdisol ®) | 5 | mg |

Lenalidomide was premixed for 10 min. together with lactose in a free-fall mixer (Turbula). After that, all the other ingredients except for magnesium stearate were added and mixed for a further 30 min. After magnesium stearate was added, final mixing continued for 2 min. The finished mixture was compressed on a rotary tableting press with biconvex punches 7 mm round. The tablets had a hardness of approx. 50-85 N. After that, the tablets could optionally be covered with a film (coating).

### Example III-2

### Dry Granulation of Amorphous Lenalidomide

| | | |
|---|---|---|
| Lenalidomide (amorphous) | 5 | mg |
| Povidon ® VA 64 | 10 | mg |
| Talcum | 1 | mg |
| Prosolv ® 90 | 90 | mg |
| Sodium bicarbonate | 30 | mg |
| Magnesium stearate | 1.3 | mg |
| Aerosil ® 300 | 0.8 | mg |

An intermediate of amorphous lenalidomide and Povidon® VA 64 was prepared by spray-drying. The intermediate was premixed for 5 min. together with half the Prosolv® 90, magnesium stearate, Aerosil® and the entire sodium bicarbonate, and compacted. After that, the material was crushed in a screen mill with 1.0 mm mesh width (Comil®) and compressed into tablets with the remaining materials.

### Example III-3

### Direct Compression of Crystalline Lenalidomide

| | | |
|---|---|---|
| Lenalidomide (form B) | 5 | mg |
| Lactose monohydrate (Tablettose 70) | 50 | mg |
| MCC (Avicel ® PH 102 ) | 55 | mg |
| Magnesium stearate | 1 | mg |
| Silica (Aerosil ® 300) | 0.5 | mg |
| Croscarmellose (Acdisol ®) | 10 | mg |

Lenalidomide was premixed together with lactose for 10 minutes in a free-fall mixer (Turbula). After that, all the other ingredients except for magnesium stearate were added and mixed for a further 30 minutes. After the addition of magnesium stearate, final mixing continued for 2 min. The finished mixture was compressed on a rotary tableting press with biconvex punches 8 mm round with a pressing force of 7.7 kN. The tablets had a hardness of approx. 66 N auf. After that, the tablets could optionally be covered with a film (coating).

1. A storage-stable intermediate, comprising amorphous lenalidomide and surface stabiliser or comprising lenalidomide and matrix material, wherein the lenalidomide is present in the form of a solid solution.

2. The storage-stable intermediate of claim 1, wherein, after storage for 3 years at 25° C. and 50% relative humidity, the proportion of crystalline lenalidomide—based on the total amount of lenalidomide—is no more than 30% by weight.

3. The storage-stable intermediate of claim 1, characterised in that the surface stabiliser, or matrix material, comprises a polymer, preferably a polymer with a glass transition temperature (Tg) higher than 25° C., or a sugar alcohol.

4. The storage-stable intermediate of claim 1, characterised in that the weight ratio of lenalidomide to surface stabiliser, or matrix material, is from about 1:1 to to about 1:10.

5. The storage-stable intermediate of claim 1 characterised in that the glass transition temperature (Tg) of the intermediate is more than 20° C.

6. The storage-stable intermediate of claim 1, characterised in that it further comprises a crystallisation inhibitor based on an inorganic salt, an organic acid, a polymer with a weight-average molecular weight of more than 500,000 g/mol or mixtures thereof.

7. The storage-stable intermediate of claim 6, wherein the crystallisation inhibitor is citric acid, ammonium chloride, Povidon K 90 or mixtures thereof.

8. The storage-stable intermediate of claim 3, wherein the polymer is polyvinyl pyrrolidone with a weight-average molecular weight of from about 10,000 to about 60,000 g/mol, a copolymer of vinyl pyrrolidone and vinyl acetate, polyethylene glycol with a weight-average molecular weight of from about 2,000 to about 10,000 g/mol, HPMC, especially with a weight-average molecular weight of from about 20,000 to about 90,000 g/mol and/or microcrystalline cellulose, especially one with a specific surface area of about 0.7 m²/g to about 1.4 m²/g.

9. The storage-stable intermediate of claim 3, wherein the sugar alcohol is selected from sorbitol, xylitol, isomalt or a mixture thereof.

10. A method of preparing a storage-stable intermediate, comprising the steps of
(i) dissolving lenalidomide and surface stabiliser, or matrix material, in a solvent or mixture of solvents, and
(ii) spraying the solution from step (i) onto a substrate core.

11. A method of preparing a storage-stable intermediate, comprising the steps of
(i) dissolving the lenalidomide, preferably the crystalline lenalidomide and the surface stabiliser or matrix material, in a solvent or mixture of solvents, and
(ii) spray-drying the solution from step (i).

12. A method of preparing a storage-stable intermediate, comprising the steps of
(i) mixing lenalidomide and surface stabiliser, or matrix material, and
(ii) melting, preferably extruding, the mixture.

13. An intermediate obtainable by a method of claim 10.

14. A pharmaceutical formulation comprising lenalidomide in the form of a storage-stable intermediate of claim 1, and optionally at least one further pharmaceutical excipient.

15. The pharmaceutical formulation of claim 14, comprising
(i) 1 to 50% by weight amorphous or molecularly disperse lenalidomide; and
(ii) 5 to 25% by weight disintegrants, based on the total weight of the dosage form.

16. A dry-granulation method of preparing a pharmaceutical formulation, comprising the steps of
(I) preparing a storage-stable intermediate of claim 1 and one or more pharmaceutical excipients;
(II) compacting the intermediate and the one or more pharmaceutical excipients into flakes; and
(III) granulating or comminuting the flakes.

17. A composition of granules obtainable by the method of claim 16.

18. The method of claim 16, wherein the granules resulting in step (III) are processed into pharmaceutical dosage forms, preferably by filling them into sachets or capsules or compressing them into tablets.

19. A tablet or capsule obtainable by the method of claim 18.

20. The tablet of claim 19, wherein the tablet has a bimodal pore size distribution.
